(19) United States
(12) Patent Application Publication
(10) Pub. No.: US 2019/0039979 A1
(43) Pub. Date: Feb. 7, 2019

(54) PROCESS FOR THE PRODUCTION OF ALKYLENE GLYCOLS

(71) Applicant: SHELL OIL COMPANY, HOUSTON, TX (US)

(72) Inventors: Evert VAN DER HEIDE, Amsterdam (NL); Pieter HUIZENGA, Amsterdam (NL); Dionysius Jacobus Maria DE VLIEGER, Amsterdam (NL)

(21) Appl. No.: 16/076,570

(22) PCT Filed: Feb. 8, 2017
(86) PCT No.: PCT/EP2017/052759

§ 371 Date: Aug. 8, 2018

(30) Foreign Application Priority Data
Feb. 9, 2016 (EP) 16154879.7

(51) Int. Cl. C07C 29/132; C07C 29/60

(52) U.S. Cl. CPC C07C 29/132; C07C 31/202; C07C 29/60

(57) ABSTRACT

The invention provides a process for the production of alkylene glycols, said process comprising providing a feed comprising at least 10 wt % of lignocellulose and/or one or more saccharides, on the basis of the overall feed, in water to a reactor; also providing a feed comprising one or more hydrogen-donating organic solvent species to the reactor; contacting the lignocellulose and/or one or more saccharides in the reactor with a retro-aldol catalyst composition at a temperature in the range of from at least 160 to at most 270° C., wherein the combined solvent system within the reactor comprises in the range of from at least 5 to at most 95 wt % of one or more hydrogen-donating organic solvent species and in the range of from at least 5 to at most 95 wt % of water.

### FIELD OF THE INVENTION

The present invention relates to a process for the production of alkylene glycols.

### BACKGROUND OF THE INVENTION

Monoethylene glycol (MEG) and monopropylene glycol (MPG) are valuable materials with a multitude of commercial applications, e.g. as heat transfer media, antifreeze, and precursors to polymers, such as polyethylene terephthalate (PET). MEG and MPG are typically made on an industrial scale by hydrolysis of the corresponding alkylene oxides, which are the oxidation products of ethylene and propylene, produced from fossil fuels.

In recent years, increased efforts have focussed on producing chemicals, including glycols, from renewable feedstocks, such as sugar-based materials. The conversion of sugars to glycols can be seen as an atom-efficient use of the starting materials with the oxygen atoms remaining intact in the desired product.

Current methods for the conversion of saccharides to glycols revolve around a retro-aldol/hydrogenation process as described in Angew. Chem. Int. Ed. 2008, 47, 8510-8513. Development of this technology has been on-going.

It is clearly desirable to maximise the yields of MEG and MPG in such processes and to deliver a process that can be carried out in a commercially viable manner. The market for MEG is generally more valuable than that for MPG, so a process particularly selective toward MEG would be advantageous.

A preferred methodology for a commercial scale process would be to use continuous flow technology, wherein feed is continuously provided to a reactor and product is continuously removed therefrom. By maintaining the flow of feed and the removal of product at the same levels, the reactor content remains at a more or less constant volume. Continuous flow processes for the production of glycols from saccharide feedstock have been described in US20110313212, CN102675045, CN102643165, WO2013015955 and CN103731258.

Processes for the conversion of saccharides to glycols generally require two catalytic species in order to catalyse the retro-aldol and hydrogenation reactions. The catalyst compositions used for the hydrogenation reactions tend to be heterogeneous. However, the catalyst compositions suitable for the retro-aldol reactions are generally homogeneous in the reaction mixture. Such homogeneous catalysts are inherently limited due to solubility constraints.

It is known that thermal degradation of reaction intermediates, such as glycolaldehyde, can occur in the conversion of saccharides to glycols. Such degradation reduces the overall yield of desired products and increases the complexity of the isolation process of said desired products. It has generally been found that carrying out the reaction with high concentrations of starting materials in a reactor exacerbates this degradation and the formation of by-products.

Typically, the conversion of saccharides to glycols has, therefore, been carried out as a continuous flow process with a high degree of back mixing using a saccharide-containing feedstock comprising a low concentration of saccharide in solvent.

Methods for maintaining a low concentration of saccharide starting material in the reaction system, while obtaining a high enough throughput and yield have been disclosed in the art, for example in co-pending application EP15198769.0. The method described in that document requires reactor system comprising a reactor vessel equipped with an external recycle loop. Saccharide-containing starting material and retro-aldol catalyst are provided to the recycle loop. As the starting material passes through the recycle loop with a short residence time, the retro-aldol reactions occur. The products of the retro-aldol reactions are then subjected to hydrogenation in the presence of a solid catalyst composition supported in the reactor vessel. A portion of the product stream is removed from the reactor vessel and the remainder is recycled back, via the recycle loop. Recycle of a portion of the product stream allows dilution of the starting material stream and efficient recycle of at least a portion of the retro-aldol catalyst composition.

Further optimisation of a process for the conversion of saccharides into glycols is always desirable. It would be preferable to carry out a continuous process to provide glycols, and particularly MEG, from saccharide-containing feedstock in as high a yield as possible. Indeed, it remains key to the development of an efficient and economically viable process for the production of alkylene glycols from saccharide-containing feedstocks to provide a process in which the concentration of starting materials can be maintained at a higher level than those viable using prior art processes.

### SUMMARY OF THE INVENTION

Accordingly, the present invention provides a process for the production of alkylene glycols, said process comprising providing a feed comprising at least 10 wt % of lignocellulose and/or one or more saccharides, on the basis of the overall feed, and water to a reactor; also providing a feed comprising one or more hydrogen-donating organic solvent species to the reactor; contacting lignocellulose and/or the one or more saccharides in the reactor with a retro-aldol catalyst composition at a temperature in the range of from at least 160 to at most 270° C., wherein the combined solvent system within the reactor comprises in the range of from at least 5 to at most 95 wt % of one or more hydrogen-donating organic solvent species and in the range of from at least 5 to at most 95 wt % of water.

### DETAILED DESCRIPTION OF THE INVENTION

The present inventors have surprisingly found that by carrying out the conversion of lignocellulose and/or saccharides to alkylene glycols in the presence of a solvent system comprising in the range of from at least 5 to at most 95 wt % of a hydrogen-donating organic solvent species and from at least 5 to at most 95 wt % water, a much higher concentration of saccharide in the solvent system can be used without detrimentally affecting the glycols yield. In fact, in many cases an increase of yield for monoethylene glycol may be obtained.

In the process of the invention, the one or more saccharides are selected from the group consisting of monosaccharides, disaccharides, oligosaccharides and polysaccharides.

Saccharides, also referred to as sugars or carbohydrates, comprise monomeric, dimeric, oligomeric and polymeric aldoses, ketoses, or combinations of aldoses and ketoses, the monomeric form comprising at least one alcohol and a carbonyl function, being described by the general formula of CₙH₂ₙOₙ (n=4, 5 or 6). Typical C₄ monosaccharides comprise erythrose and threose, typical C₅ saccharide monomers include xylose and arabinose and typical C₆ sugars comprise aldoses like glucose, mannose and galactose, while a common C₆ ketose is fructose. Examples of dimeric saccharides, comprising similar or different monomeric saccharides, include sucrose, maltose and cellobiose. Saccharide oligomers are present in corn syrup. Polymeric saccharides include cellulose, starch, glycogen, hemicellulose, chitin, and mixtures thereof.

If the one or more saccharides comprise oligosaccharides or polysaccharides, it is preferable that they are subjected to pre-treatment before being fed to the process in a form that can be converted in the process of the present invention. Suitable pre-treatment methods are known in the art and one or more may be selected from the group including, but not limited to, sizing, drying, grinding, hot water treatment, steam treatment, hydrolysis, pyrolysis, thermal treatment, chemical treatment, biological treatment. However, after said pre-treatment, the starting material still comprises mainly monomeric and/or oligomeric saccharides. Said saccharides are, preferably, soluble in the reaction solvent.

In one preferred embodiment of the invention, the one or more saccharides used in the process of the invention, after any pre-treatment, comprise saccharides selected from starch and/or hydrolysed starch. Hydrolysed starch comprises glucose, sucrose, maltose and oligomeric forms of glucose.

In another preferred embodiment of the invention, the one or more saccharides comprise cellulose, hemi-cellulose, saccharides derived from lignocellulose, and/or sugars derived therefrom. In this embodiment, the one or more saccharides are preferably derived from softwood.

The lignocellulose and/or one or more saccharides are provided to the reactor as a feed comprising at least 10 wt %, preferably at least 12 wt %, more preferably at least 15 wt %, even more preferably at least 20 wt %, most preferably at least 40 wt %, of said lignocellulose and/or one or more saccharides in water. Said lignocellulose and/or one or more saccharides are suitably present as a solution, a suspension or a slurry in the water.

A feed comprising one or more hydrogen-donating organic solvent species is also provided to the reactor. This feed may form part of the same feed as the one or more saccharides in water. Alternatively this feed may be mixed with that stream before being provided to the reactor or at the time of being provided to the reactor.

These feeds and any others, including a source of the retro-aldol catalyst composition, optionally in a solvent, combine in the reactor to form the reactor contents. There is, therefore, a combined solvent system within the reactor. Said solvent system comprises in the range of from at least 5 to at most 95 wt % of one or more hydrogen-donating organic solvent species and in the range of from at least 5 to at most 95 wt % water. Preferably, the solvent system comprises at least 10 wt %, more preferably at least 20 wt %, even more preferably at least 40 wt % of one or more hydrogen-donating organic solvent species. Also preferably, the solvent system comprises at most 90 wt %, more preferably at most 80 wt %, more preferably at most 75 wt % of the one or more hydrogen-donating organic solvent species. Preferably, the solvent system comprises at least 10 wt %, more preferably at least 20 wt %, even more preferably at least 25 wt % of water. Also preferably, the solvent system comprises at most 90 wt %, more preferably at most 80 wt %, more preferably at most 60 wt % of water.

The term ‘hydrogen-donating’ when referring to the organic solvent species as used herein takes its usual meaning. That is, it refers to the ability of the species to donate hydrogen to another species in a reaction mixture under the reaction conditions. The bond between the donating species and the hydrogen atom is broken. It will be readily apparent to the skilled person that this does not cover ‘hydrogen bond donation’ in which one molecule donates a hydrogen bond to another molecule while the covalent bond between the hydrogen atom and the first molecule remains intact.

Preferably, the hydrogen-donating organic solvent species is selected from the group of secondary alcohols, glycols, sugar alcohols, hydroquinone and formic acid. Preferable secondary alcohols include isopropyl alcohol and 2-butanol. Preferable sugar alcohols include glycerol, erythritol, threitol, sorbitol, xylitol. Preferable glycols include 1,2-butanediol and 2,3-butanediol.

In the process of the invention, the one or more saccharides are contacted with a retro-aldol catalyst composition. Said retro-aldol catalyst composition preferably comprises one or more compound, complex or elemental material comprising tungsten, molybdenum, vanadium, niobium, chromium, titanium, tin or zirconium. More preferably the retro-aldol catalyst composition comprises one or more material selected from the list consisting of tungstic acid, molybdic acid, ammonium tungstate, ammonium metatungstate, ammonium paratungstate, silver tungstate, zinc tungstate, zirconium tungstate, tungstate compounds comprising at least one Group 1 or 2 element, metatungstate compounds comprising at least one Group 1 or 2 element, paratungstate compounds comprising at least one Group 1 or 2 element, heteropoly compounds of tungsten including group 1 phosphotungstates, heteropoly compounds of molybdenum, tungsten oxides, molybdenum oxides, vanadium oxides, metavanadates, chromium oxides, chromium sulfate, titanium ethoxide, zirconium acetate, zirconium carbonate, zirconium hydroxide, niobium oxides, niobium ethoxide, and combinations thereof. The metal component is in a form other than a carbide, nitride, or phosphide. Preferably, the retro-aldol catalyst composition comprises one or more compound, complex or elemental material selected from those containing tungsten or molybdenum.

The retro-aldol catalyst composition may be present as a heterogeneous or a homogeneous catalyst composition. In one embodiment, the retro-aldol catalyst composition is heterogeneous with respect to the reaction mixture and is supported in a reactor. In a preferred embodiment, the retro-aldol catalyst composition is homogeneous with respect to the reaction mixture. In this embodiment, the retro-aldol catalyst composition and any components contained therein, may be fed into the reactor in which the process is carried out as required in a continuous or discontinuous manner during the process for the preparation of alkylene glycols. Typically, in this embodiment, the retro-aldol catalyst composition may be provided to the reactor in a solvent (for example, water, hydrocarbon heavies stream, hydrogen-donating solvent or mixtures thereof). This solvent will form part of the solvent system in the reactor. Optionally, the catalyst may be co-fed with or form part of one of the other streams provided to the reactor.

The weight ratio of the retro-aldol catalyst composition (based on the amount of metal in said composition) to sugar in the feed is suitably in the range of from 1:1 to 1:1000.

The lignocellulose and/or one or more saccharides are contacted with the retro-aldol catalyst composition at a temperature in the range of from at least 160 to at most 270° C. Preferably, the temperature is at least 170° C., most preferably at least 190° C. Also preferably, the temperature is at most 250° C.

The pressure in the reactor in which the lignocellulose and/or one or more saccharides are contacted with the retro-aldol catalyst composition is at least 1 MPa, preferably at least 2 MPa, most preferably at least 3 MPa. The pressure is preferably at most 18 MPa, more preferably at most 15 MPa, most preferably at most 12 MPa.

The pH in the reaction mixture when the lignocellulose and/or one or more saccharides are contacted with the retro-aldol catalyst composition is preferably at least 2.0, more preferably at least 2.5. The pH in the reaction mixture is preferably at most 8.0, more preferably at most 6.0. Optionally, the pH may be maintained by using a buffer. Examples of suitable buffers include, but are not limited to, acetate buffers, phosphate buffers, lactate buffers, glycolate buffers, citrate buffers and buffers of other organic acids.

As well as contacting lignocellulose and/or one or more saccharides with a retro-aldol catalyst composition in a retro-aldol step, a typical process for the production of alkylene glycols also involves a hydrogenation step. Said hydrogenation step involves reaction with hydrogen in the presence of a hydrogenation catalyst composition.

The hydrogenation catalyst composition is preferably heterogeneous and is retained or supported within a reactor. Further, said hydrogenation catalyst composition also preferably comprises one or more materials selected from transition metals from groups 8, 9 or 10 or compounds thereof, with catalytic hydrogenation capabilities.

More preferably, the hydrogenation catalyst composition comprises one or more metals selected from the list consisting of iron, cobalt, nickel, ruthenium, rhodium, palladium, iridium and platinum. This metal or metals may be present in elemental form or as compounds. It is also suitable that this component is present in chemical combination with one or more other ingredients in the hydrogenation catalyst composition. It is required that the hydrogenation catalyst composition has catalytic hydrogenation capabilities and it is capable of catalysing the hydrogenation of material present in the reactor.

In one embodiment, the hydrogenation catalyst composition comprises metals supported on a solid support. In this embodiment, the solid supports may be in the form of a powder or in the form of regular or irregular shapes such as spheres, extrudates, pills, pellets, tablets, monolithic structures. Alternatively, the solid supports may be present as surface coatings, for examples on the surfaces of tubes or heat exchangers. Suitable solid support materials are those known to the skilled person and include, but are not limited to aluminas, silicas, zirconium oxide, magnesium oxide, zinc oxide, titanium oxide, carbon, activated carbon, zeolites, clays, silica alumina and mixtures thereof.

Alternatively, the heterogeneous hydrogenation catalyst composition may be present as Raney material, such as Raney nickel or Raney ruthenium, preferably present in a pelletised form.

The heterogeneous hydrogenation catalyst composition is suitably preloaded into the reactor before the reaction is started.

The hydrogenation step and the retro-aldol step may be carried out in a ‘one pot’ process wherein both catalyst compositions are present simultaneously in a single reactor system. Alternatively, the retro-aldol step may be carried out in a first reactor or reaction zone and then the hydrogenation step is carried out in a second reactor or reaction zone. In this embodiment, the hydrogenation catalyst is only present in this second reactor or reactor zone. Further, in this embodiment wherein first and second reaction zones or reactors are present, said reaction zones or reactors are physically distinct from one another. Each reaction zone may be an individual reactor or reactor vessel or the zones may be contained within one reactor vessel.

The hydrogenation step and, optionally, the retro-aldol step of the process of the present invention take place in the presence of hydrogen. Preferably, both steps (if carried out) take place in the absence of air or oxygen. In order to achieve this, it is preferable that the atmosphere under which the process takes place (e.g. in the reaction zones) be evacuated and replaced with first an inert gas, e.g. nitrogen or argon, and then hydrogen repeatedly, after loading of any initial contents, before the reaction starts.

A product stream is removed from the hydrogenation step. At least a portion of the product stream is provided for separation and purification of the glycols contained therein. Steps for purification and separation may include solvent removal, catalyst separation, distillation and/or extraction in order to provide the desired glycol products.

Typically, said product stream is separated into at least a glycol product stream and a hydrocarbon heavies stream. The hydrocarbon heavies stream will contain sugar alcohols, other heavy organics and catalyst components. At least a portion of this stream may be recycled to the process, with or without separation of the catalyst components. In one embodiment of the invention, glycerol present in this stream may be separated and used as at least a portion of the hydrogen-donating organic solvent species in the solvent system in the reactor.

The present invention is further illustrated in the following Examples.

### Examples 1 and 2

A hastelloy (C22) autoclave of 100 ml total volume (50 ml liquid hold-up) was loaded with 3.5 g Raney Ni (type 2800). The Raney Ni catalyst was activated and the reactor was brought to steady state reaction conditions. The reaction temperature was 220° C. and total pressure was 12 MPa. The gas phase comprised mainly hydrogen and water in equilibrium with the liquid phase. The system was run at a stable state with a pH of the reactor effluent of 4.1. Under steady state reaction conditions, H₂ gas was fed to the reactor at 3 L/h STP (standard temperature and pressure). An aqueous solution of 7600 ppmw sodium metatungstate, 4.5 g/L sodium acetate and 3.0 g/L acetic acid was fed to the reactor at a rate of 20 g/hr via a first feedline. Simultaneously, a second feedline was used to feed a 20 wt % glucose solution in water to the reactor at a rate of 20 g/hr. Both feeds resulted in a total reactor feed of 10 wt % glucose, 3800 ppmw sodium metatungstate, 2.25 g/L sodium acetate and 1.5 g/L acetic acid to the reactor at a rate of 40 g/hr. Residence time in the reactor was 75 minutes. The pH during the run was 4.11. The results of this run are shown in Table 1 for Example 1 (comparative Example).

At a certain moment, under steady state conditions, glycerol (a hydrogen-donating organic solvent species) was added to the glucose solution (20 wt % glucose and 20 wt % glycerol). This resulted in a feed of 10 wt % glucose, 10 wt % glycerol, 3800 ppmw sodium metatungstate, 2.25 g/L sodium acetate and 1.5 g/L acetic acid to the reactor at a rate of 40 g/hr. The pH during the run was 4.19. No other reaction parameters were changed. The results of this run are recorded in Table 2 for Example 2 (or the invention)

For each run, the reactor effluent was analysed by HPLC and the product yields are tabulated in Table 1.

TABLE 1

| | | Sor- | C4sugar | Glyc- | | | 1,2- | 1,2- | |
|---|---|---|---|---|---|---|---|---|---|
| | Glucose | bitol | alcohol | erol | MEG | MPG | BDO | HDO | Total |
| | (%) | (%) | (%) | (%) | (%) | (%) | (%) | (%) | (%) |
| 1 | 0.3 | 31.5 | 9.2 | 5.6 | 31.8 | 5.3 | 2.7 | 0.4 | 86.9 |
| 2 | 0.9 | 29.0 | 9.2 | 3.5 | 39.1 | 7.4 | 2.8 | 0.4 | 92.3 |

The glycerol yield given in Table 1 is the yield after subtraction of the amount of glycerol added to the process.

The MEG yields increased from 31.8 to 39.1 when glycerol was co-fed. Sorbitol formation was a bit lower when glycerol was co-fed and could account for 1.9% more MEG make. The total yield of desirable components increased by 5.4% due to the increase in MEG yield, indicating that less MEG intermediates were degrading to undesired products. Some additional MPG (2.1%) was also formed.

### Example 3 to 6

A 60 ml Hastelloy C22 autoclave (Premex) was loaded with 30 ml of a water and glycerol mixture (50 wt %/50 wt %), 300 mg glucose, 30 mg sodium phosphotungstate (Na₃PW₁₂O₄₀) and 90.1 mg 1% w ruthenium on silica (Ru(1.0)/SiO₂) catalyst (as set out in Table 2). The reactor was closed, the gas phase replaced by nitrogen, then hydrogen, pressurized to 7.0 MPa pressure, heated to 195° C. for 90 minutes where a total pressure of 9.4 MPa was reached, and cooled down. The products were analysed by gas chromatography.

For Examples 4 to 6, Example 3 was repeated, except that the water and glycerol mixture had compositions as indicated in Table 2.

Results for Examples 3 to 6 are indicated in Table 3.

TABLE 2

| | Water | Glycerol | Glucose | Na₃PW₁₂O₄₀ | Ru (1.0)/SiO₂ |
|---|---|---|---|---|---|
| | (% wt) | (% wt) | (mg) | (mg) | (mg) |
| 3 | 50.0 | 50.0 | 300 | 30.0 | 90.1 |
| 4 | 30.0 | 70.0 | 300 | 30.6 | 90.4 |
| 5 | 10.0 | 90.0 | 300 | 30.1 | 90.2 |
| 6 | 5.0 | 95.0 | 300 | 30.1 | 90.2 |

TABLE 3

| | MEG | MPG | HA | 1,2-BDO | 1H2BO |
|---|---|---|---|---|---|
| | (% wt) | (% wt) | (% wt) | (% wt) | (% wt) |
| 3 | 41.0 | 9.6 | 2.9 | 2.5 | 1.2 |
| 4 | 39.8 | 12.0 | 2.6 | 2.1 | 0.8 |
| 5 | 38.1 | 13.8 | 2.8 | 2.8 | 0.7 |
| 6 | 29.0 | 14.0 | 3.5 | 2.5 | 0.7 |

MEG: monoethylene glycol;
MPG: monopropylene glycol;
HA: hydroxyacetone;
1,2-BDO: 1,2-butanediol;
1H2BO: 1-hydroxy-2-butanone.

1. A process for the production of alkylene glycols, said process comprising providing a feed comprising at least 10 wt % of lignocellulose and/or one or more saccharides, on the basis of the overall feed, in water to a reactor; also providing a feed comprising one or more hydrogen-donating organic solvent species to the reactor; contacting the lignocellulose and/or one or more saccharides in the reactor with a retro-aldol catalyst composition at a temperature in the range of from at least 160 to at most 270° C., wherein the combined solvent system within the reactor comprises in the range of from at least 5 to at most 95 wt % of one or more hydrogen-donating organic solvent species and in the range of from at least 5 to at most 95 wt % of water.

2. The process according to claim 1, wherein the hydrogen-donating organic solvent species is selected from the group consisting of secondary alcohols, glycols, hydroquinone, formic acid and sugar alcohols.

3. The process as claimed in claim 2, wherein the hydrogen-donating organic solvent species is selected from isopropyl alcohol, glycerol, erythritol, threitol, sorbitol, xylitol, 2-butanol, 1,2-butanediol, 2,3-butanediol, hydroquinone and formic acid.

4. The process as claimed in claim 1, wherein the one or more saccharides comprises starch and/or hydrolysed starch.

5. The process as claimed in claim 1, wherein the one or more saccharides comprises cellulose, hemi-cellulose, saccharides derived from lignocellulose, and/or sugars derived therefrom.

6. The process as claimed in claim 5, wherein the one or more saccharides are derived from softwood.

7. The process as claimed in claim 1, wherein the retro-aldol catalyst composition comprises one or more compound, complex or elemental material selected from those containing tungsten or molybdenum.

8. The process as claimed in claim 1, wherein the process also comprises a hydrogenation step, which involves reaction with hydrogen in the presence of a hydrogenation catalyst composition.

9. The process as claimed in claim 8, wherein both the retro-aldol catalyst composition and the hydrogenation catalyst composition are present simultaneously in a single reactor system.

10. The process as claimed in claim 8, wherein the retro-aldol step is carried out in a first reaction zone and then the hydrogenation step is carried out in a second reaction zone.

11. The process as claimed in claim 8, wherein a product stream is removed from the hydrogenation step and at least a portion of the product stream is separated into at least a glycol product stream and a hydrocarbon heavies process stream.

12. The process as claimed in claim 11, wherein at least a portion of the hydrocarbon heavies stream comprising sugar alcohols is recycled to the process as at least a portion of the hydrogen-donating organic solvent species in the solvent.

13. The process according to claim 12, wherein the sugar alcohol comprises glycerol.
